(19) United States
(12) Patent Application Publication
(10) Pub. No.: US 2018/0200508 A1
(43) Pub. Date: Jul. 19, 2018

(54) DEVICE FOR PACEMAKER LEAD PLACEMENT AND METHODS THEREOF

(71) Applicant: LSI Solutions, Inc., Victor, NY (US)

(72) Inventor: Jude S. Sauer, Pittsford, NY (US)

(21) Appl. No.: 15/831,121

(22) Filed: Dec. 4, 2017

Related U.S. Application Data
(60) Provisional application No. 62/429,679, filed on Dec. 2, 2016.

(51) Int. Cl. A61N 1/05; A61B 17/34; A61N 1/362; A61N 1/372

(52) U.S. Cl. CPC A61N 1/0592; A61B 17/3468; A61N 1/372; A61N 1/362; A61N 1/059

(57) ABSTRACT

A device for pacemaker lead placement is disclosed. The device has a device tip having a tissue bite area, ferrule holders, and needle tips. The device also has a lead end rest. The device also has a pacemaker lead end situated on the lead end rest, the pacemaker lead end having first and second anchor suture holes. The device further has an anchor suture with ferrules at respective ends of the anchor suture, wherein the ferrules of the anchor suture are passed through the anchor suture holes and into communication with the ferrule holders.

### RELATED APPLICATION

This application claims priority to U.S. Provisional Patent Application No. 62/429,679 filed Dec. 2, 2016 and entitled, “DEVICE FOR PEDIATRIC LEAD PLACEMENT AND METHODS THEREOF”. The 62/429,679 application is hereby incorporated by reference in its entirety.

### FIELD

The claimed invention relates to surgical devices, and more specifically to surgical devices which may be used for the placement of a pacemaker lead onto the heart of a pediatric patient using minimally invasive surgical techniques.

### BACKGROUND

Pacemaker therapy in children involves unique issues regarding patient size, growth, development, and possible presence of congenital heart disease. Traditionally, pacing leads can be implanted via the transvenous (endocardial) or surgical (epicardial) route. The choice of route has been dependent upon the size of the patient, anatomy, and surgical procedures performed that can affect the access to certain cardiac structures.

Traditionally, epicardial lead placement has been recommended in patients less than 15 kg, patients with intracardiac shunt lesions, patients with limited access to the atrium or the ventricle (e.g. patients with single ventricular physiology post Fontan palliation), and patients with prosthetic tricuspid valves. Traditionally, epicardial lead implantation has required a sternotomy or thoracotomy or subxiphoid approach, and is associated with higher chronic stimulation threshold, higher lead failures and fractures, and early depletion of battery life. However, it preserves the venous access for future use.

Endocardial (Transvenous route) lead implantation has been preferred in most pediatric patients except for those situations referred above. Endocardial lead placement has offered the advantages of avoidance of thoracotomy, lower pacing thresholds, and a lower incidence of lead fractures. However, its disadvantages include a greater risk of lead dislodgment, venous occlusion, embolic vascular events, and endocarditis.

It would be desirable to have an effective, easy to use device for pediatric lead placement that avoids both the need for a sternotomy/thoracotomy while also avoiding endocardial lead placement entirely.

### BRIEF DESCRIPTION OF THE DRAWINGS

FIGS. 1A-1C show different views of one embodiment of a pediatric scope port.

FIGS. 2A, 2B, 2C, 2D, 2E, and 2F are front (proximal side), left, right, rear (distal side), top, and bottom elevational views, respectively of the pediatric scope port of FIGS. 1A-1C.

FIG. 3 schematically illustrates a patient with a heart in need of pacemaker lead placement.

FIG. 4 schematically illustrates the pediatric scope port of FIGS. 1A-1C being slid into an incision towards the pericardium of the patient from FIG. 3.

FIG. 5 schematically illustrates a viewing scope inserted into an opening of a scope port cannula in the pediatric scope port of FIG. 4.

FIG. 6 schematically illustrates the embodied pediatric lead port of FIGS. 11A-11C inserted into a patient incision along with the pediatric scope port of FIG. 4.

FIG. 7 illustrates one embodiment of a horizontal mattress suture stitch.

FIG. 8A illustrates a bottom view one embodiment of a pediatric pacemaker lead end having and anchoring suture and first and second coupling sutures.

FIG. 8B illustrates a top view of the embodied pediatric pacemaker lead end of FIG. 8A.

FIG. 8C illustrates the pacemaker lead end of FIGS. 8A, 8B secured to heart tissue with a mechanical knot.

FIG. 8D illustrates an embodiment of a wiring casing having multiple pacemaker leads.

FIGS. 9A-9C show different views of one embodiment of a lead brace.

FIG. 10A illustrates one embodiment of a minimally invasive surgical suturing device for placing an anchor stitch for a pacemaker lead and delivering the pacemaker lead end to the space around the heart.

FIGS. 10B-10D show the distal end of the minimally invasive surgical suturing device from a variety of different perspectives

FIGS. 11A-11C show different views of one embodiment of a pediatric lead cannula.

FIGS. 12A, 12B, 12C, 12D, 12E, 12F are front (proximal side), left, right, rear (distal side), top, and bottom elevation views, respectively, of the pediatric lead cannula of FIGS. 11A-11C.

FIGS. 13-20 illustrate the surgical suturing device of FIGS. 10A-10D from a variety of different perspectives.

### DETAILED DESCRIPTION

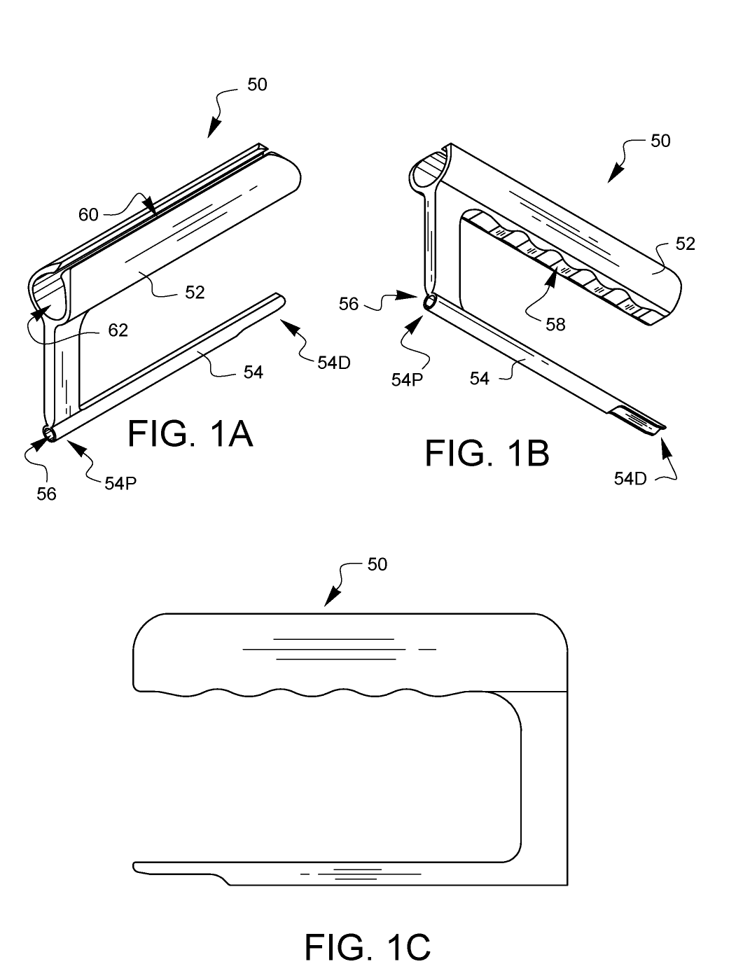

The figures included in this specification are not necessarily drawn to scale. The devices and methods disclosed herein contemplate one or more ports as part of a pediatric lead system. One such port could be a pediatric scope port 50, such as the one shown in top and bottom perspective views (FIGS. 1A and 1B) as well as in a side view in FIG. 1C.

The pediatric scope port 50 has a handle 52 coupled to and spaced from a scope cannula 54. The scope cannula 54 has a proximal end 54P and a distal end 54D. The handle 52 is coupled to the proximal end 54P of the scope cannula. An endoscopic viewing scope or the like may be introduced through a proximal opening 56 in the scope cannula 54. The handle 52 may have a grip surface 58 to facilitate holding by a hand. The handle 52 may also have a cable access slot 60 which leads to an open handle interior 62. Cables from a viewing scope, such as the light source cable and/or the imaging cable, may be routed into the open handle interior 62 via the cable access slot 60 for efficient cable management as desired.

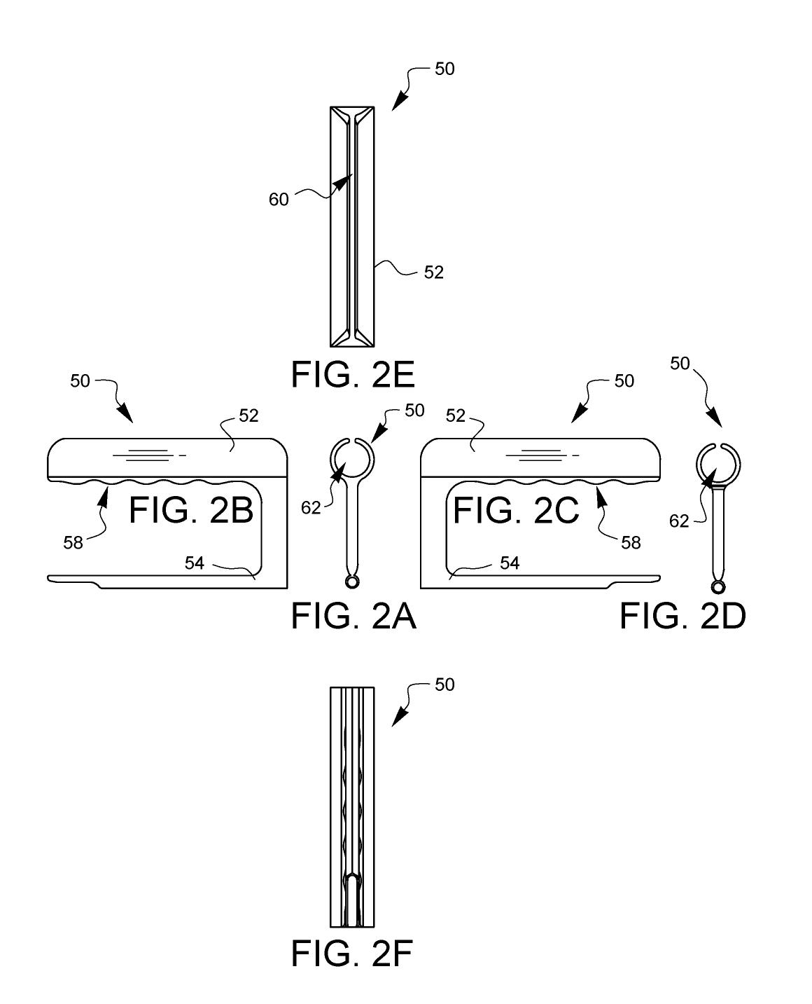

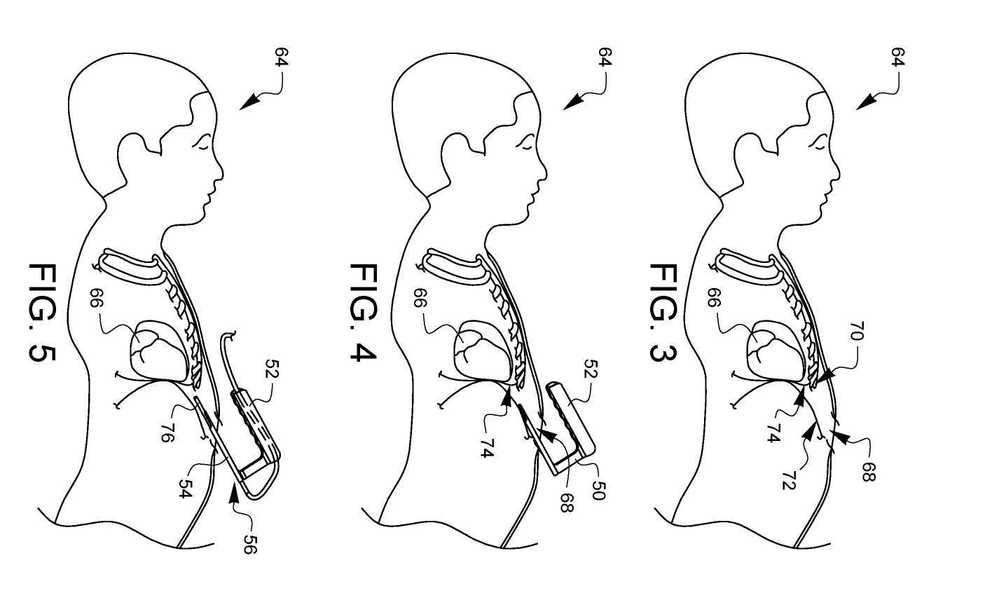

FIGS. 2A, 2B, 2C, 2D, 2E, and 2F are front (proximal side), left, right, rear (distal side), top, and bottom elevational views, respectively of the pediatric scope port 50.

Consider FIG. 3 which offers a schematic side view of a patient 64 with a heart 66 in need of pacemaker lead placement. A small incision 68 is made below the xiphoid 70 and above the diaphragm 72. If desired, a finger may be used to explore into this incision in order to create a channel towards the pericardium 74, just below the xiphoid 70.

The pediatric scope port 50 may be slid into the incision 68 towards the pericardium 74 as illustrated schematically in FIG. 4.

A viewing scope 76 may be inserted into the proximal opening 56 in the scope port cannula 54 to provide the surgeon with a view of the channel leading to the pericardium 74 as well as the pericardium 74 itself as schematically illustrated in FIG. 5.

Minimally invasive surgical tools may also be introduced into the incision in order to create an opening in the pericardium, thereby exposing the heart without the need for a sternotomy, a thoracotomy, or endocardial methods. As desired, the handle 52 on the pediatric scope port 50 may be used by a surgical assistant to pull in an anterior direction. This will cause the top side of the scope cannula 54 to pull tissue above the scope cannula in an anterior direction, thereby opening up the access passage as needed for other instruments.

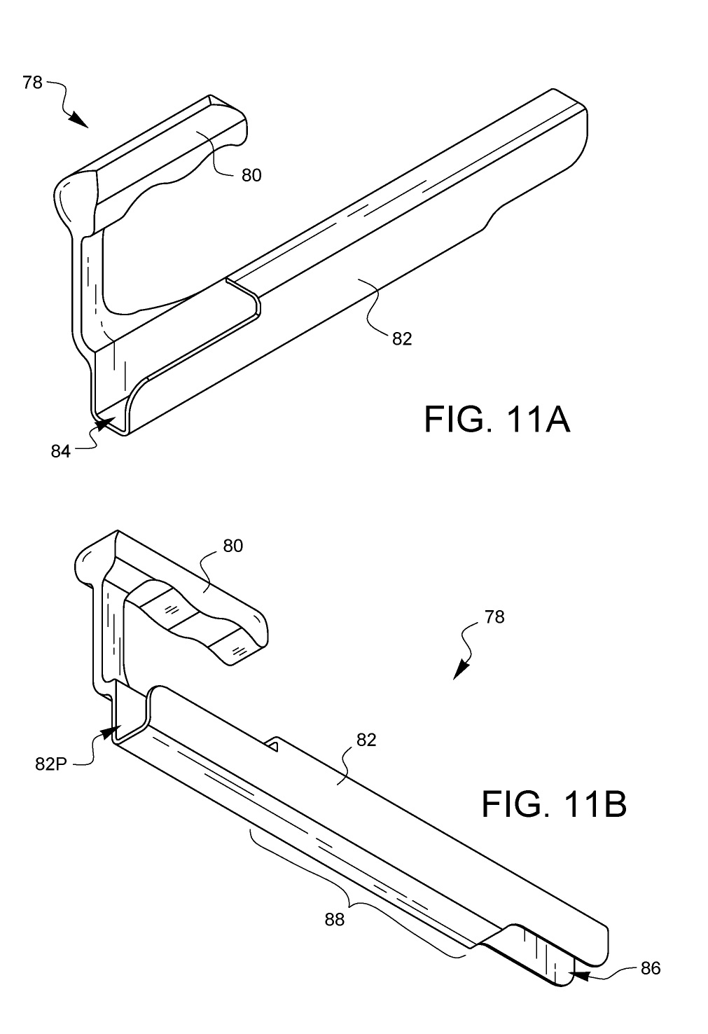

It may be helpful to have a pediatric lead cannula 78 which can be inserted next to the pediatric scope port 50. One embodiment of the pediatric lead cannula 78 is shown in the top and bottom perspective views of FIGS. 11A and 11B as well as FIG. 11C.

The pediatric lead cannula 78 has a handle 80 coupled to the proximal 82P end of a cannula portion 82. The proximal opening 84 of the cannula portion 82 is bounded on three sides, as is the distal opening 86 of the cannula. The main body 88 of the cannula portion 82 is bounded on four sides and is sized to provide clearance for a minimally invasive surgical suturing device to be passed through the cannula portion 82″.

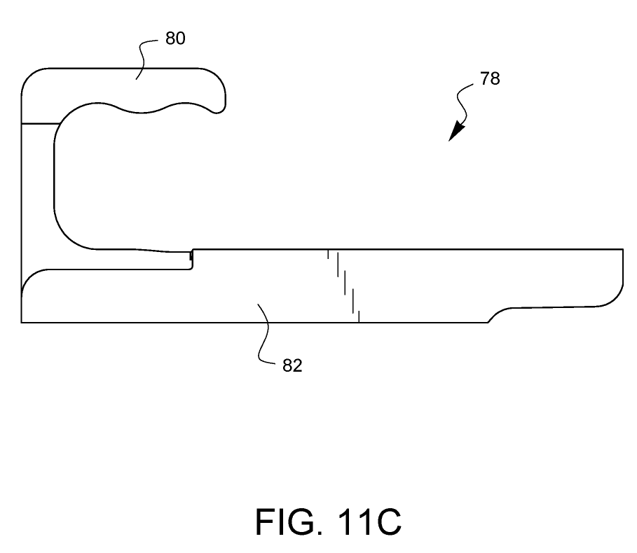

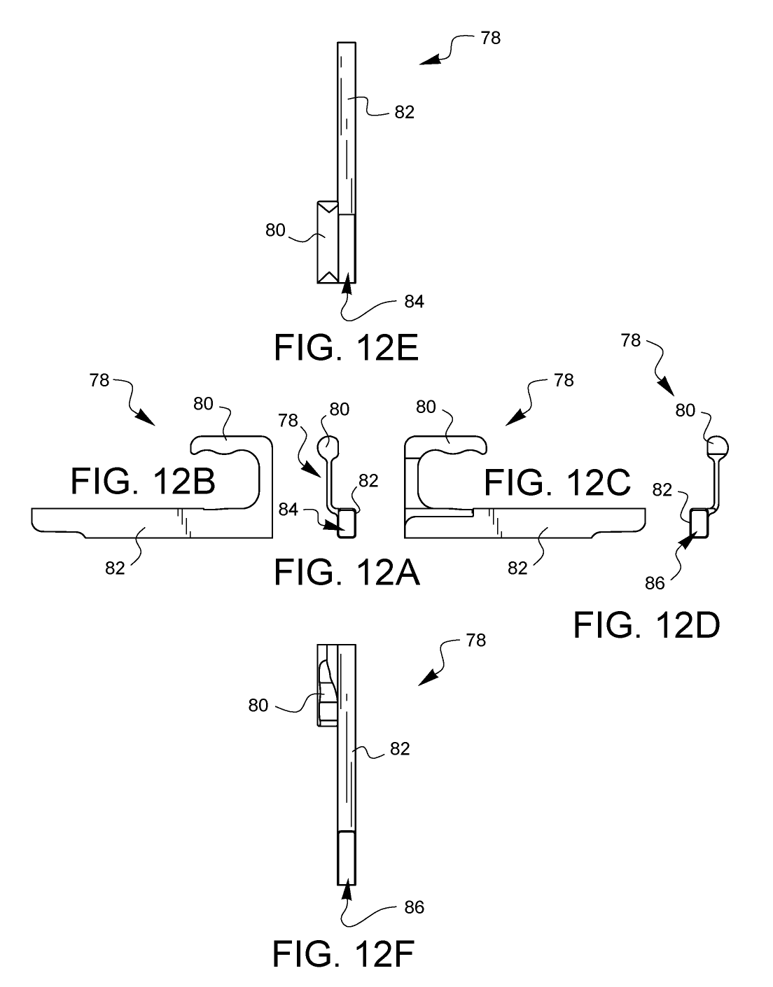

FIGS. 12A, 12B, 12C, 12D, 12E, 12F are front (proximal side), left, right, rear (distal side), top, and bottom elevation views, respectively, of the pediatric lead cannula 78.

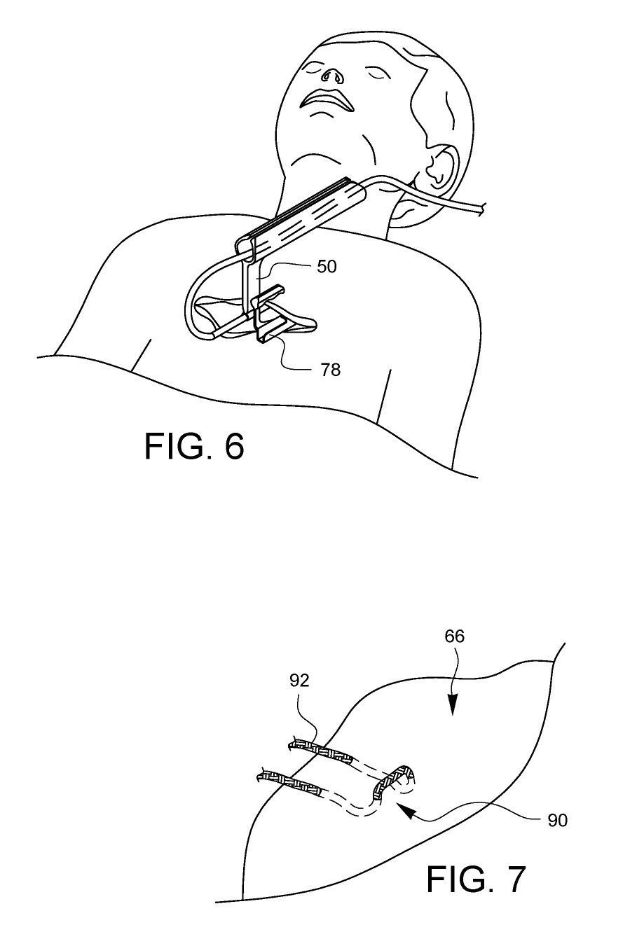

FIG. 6 schematically illustrates a pediatric scope port 50 and the pediatric lead cannula 78 installed in the incision.

A horizontal mattress suture stitch 90, or a whatever stitch desired and deemed appropriate by the surgeon, will need to be made in the heart 66 where the pediatric lead is desired to be placed. The stitch 90 is made with suture 92. FIG. 7 schematically illustrates this type of stitch 90. This can be done with a needle driver or preferably with an automated suturing tool.

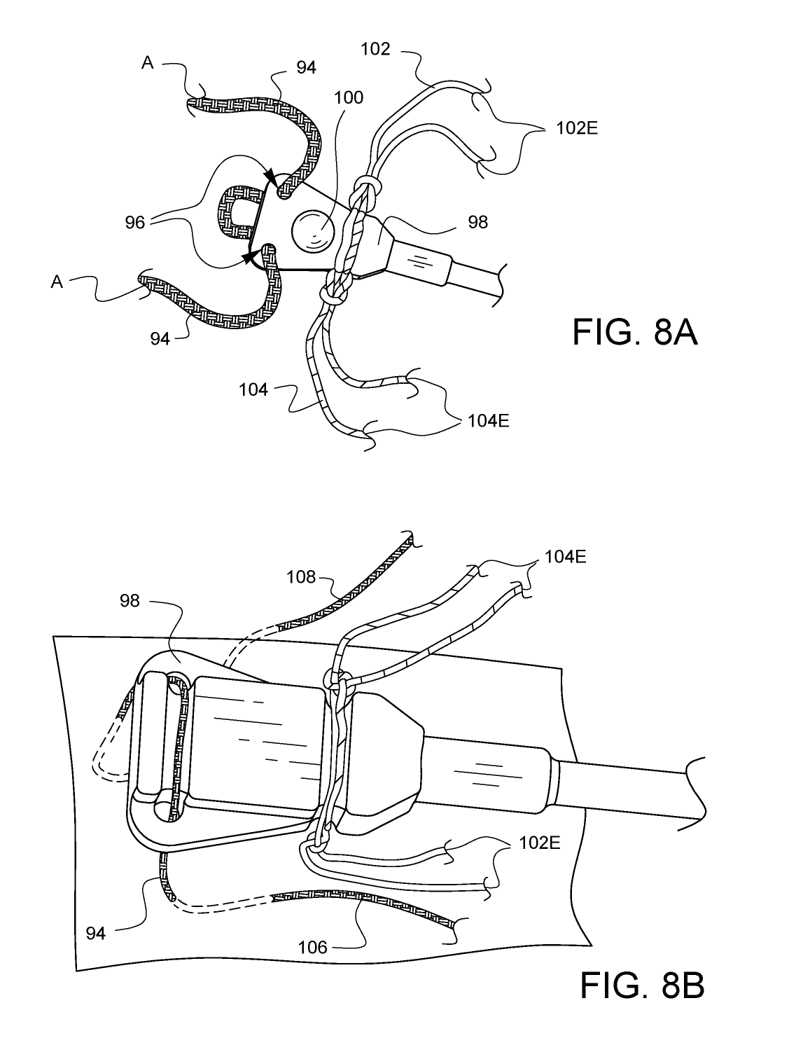

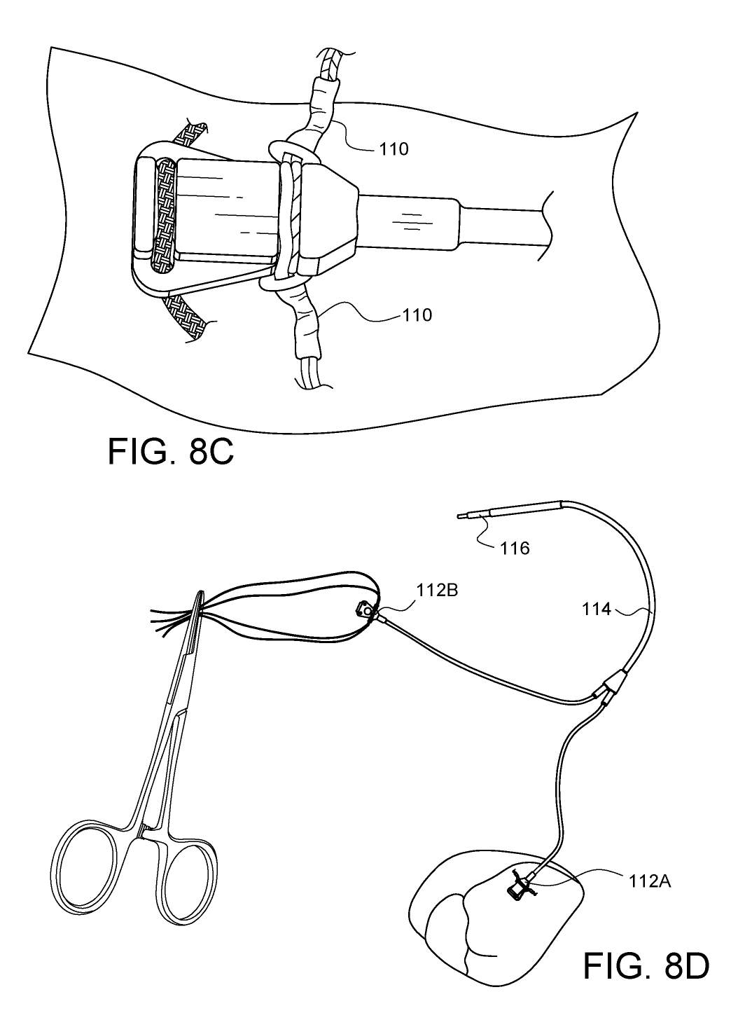

The suture used to make this stitch in the heart may be called the anchor suture 94, and its ends should be routed as shown in FIG. 8A through anchor suture holes 96 in the pacemaker lead end 98. In this embodiment, the anchor suture holes 96 are located distally when compared to the lead electrode 100 on the pacemaker lead end 98. The lead electrode 100 is the portion of the pacemaker lead which will need to make good contact with the heart.

As shown in FIG. 8A, a first coupling suture 102 is coupled to the lead end 98 at a position proximal to the lead electrode 100. A second coupling suture 104 is also coupled to the lead end 98 at a position proximal to the lead electrode 100. In our embodiment, the first and second coupling sutures 102, 104 are colored differently and the first coupling suture 102 points in one direction while the second coupling suture 104 points in a substantially opposite direction. In this embodiment, the first coupling suture 102 was attached by knotting it to the lead end 98. Similarly, the second coupling suture 104 was attached by knotting it to the lead end 98. As a result, the first coupling suture 102 has two suture ends 102E which extend from the lead end 98. Similarly, the second coupling suture 104 has two suture ends 104E which extend from the lead end 98. Preferably, the anchor suture 94 is a different color from either of the coupling sutures 102, 104.

The lead end 98 may be routed through the pediatric lead cannula 78 along the anchor suture towards the heart 66. The lead electrode 100 should face the heart. As illustrated in FIG. 8B, a first end 106 of the anchor suture 96 is aligned with the suture ends 102E of the first coupling suture 102. Similarly, a second end 108 of the anchor suture 96 is aligned with the suture ends 104E of the second coupling suture 104.

The first end 106 of the anchor suture 96 and the ends 102E of the first coupling suture 102 may be secured together against the side of the pacemaker lead end 98. Similarly, the second end 108 of the anchor suture 96 and the ends 104E of the second coupling suture 104 may be secured together against the other side of the pediatric lead 98. While handtied knots may be used, mechanical knots may be preferable. For example, FIG. 8C shows the pacemaker lead end 98 secured with the COR-KNOT® QUICK LOAD® mechanical knots 110 available from LSI Solutions, Inc. of Victor, N.Y. (See www.lsisolutions.com). Secure knots, in this case, mechanical knots 110, maintain the lead electrode 100 against the tissue of the heart.

There will often be more than one pacemaker lead which will need to be attached to the heart. For example, like the pacemaker leads 112A, 112B shown in FIG. 8D, the first pacemaker lead 112A and the second pacemaker lead 112B may branch off from a single wiring casing 114 which has a connector 116 compatible with whichever pacemaker has been selected for the patient.

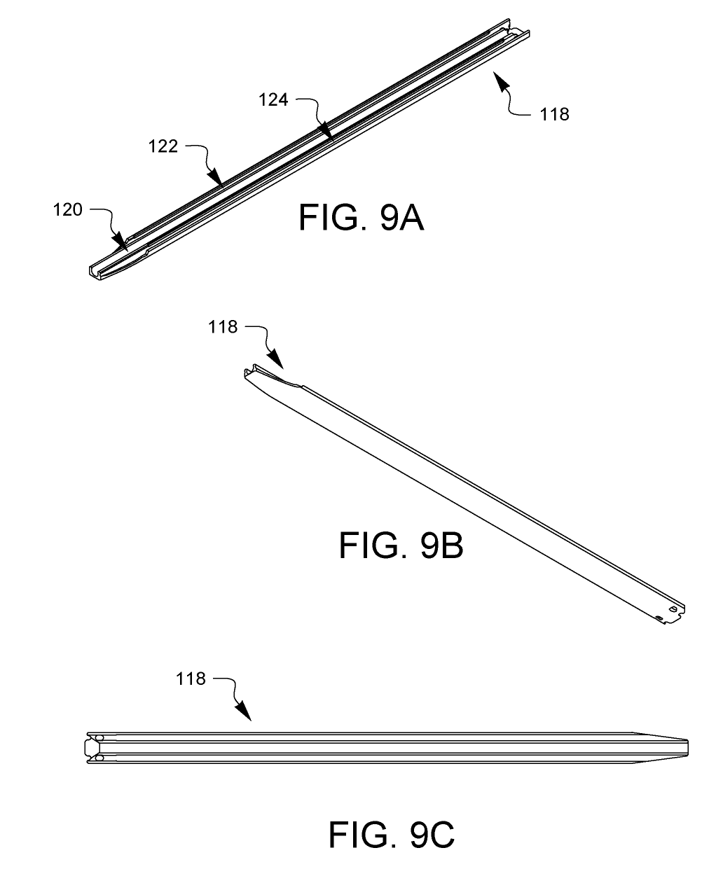

The pacemaker leads may be highly flexible, which can cause difficulties when trying to insert the leads through the patient's incision and access path through pericardium and into the space around the heart. One or more lead braces 118 may be provided to support the pacemaker lead 112A, 112B while it is being brought into position near the heart. One embodiment of a lead brace 118 is illustrated below in the top and bottom perspective views of FIGS. 9A and 9B, as well as in the top view of FIG. 9C.

The lead brace 118 has a center channel 120 which is sized to removably hold and support the pediatric lead 112A or 112B. Adjacent to the center channel 120 are two outside channels 122, 124. For purposes of suture management, the ends of the first coupling suture 102E attached to the lead end 98 are preferably threaded through a first tube (to be shown later). One of the outside channels 122 is sized to removably hold and support the tube through which the ends 102E of the first coupling suture 102 run. Similarly, the ends 104E of the second coupling suture 104 attached to the lead end 98 are preferably threaded through a second tube (to be shown later). The other outside channel 124 is sized to removably hold and support the tube through which the ends 104E of the second coupling suture 104 run. The lead brace 118 can help to overcome any tissue resistance when delivering the lead end 98 to the heart, and then the lead brace 118 can be removed. The lead brace 118 is also preferably sized to fit within the lead cannula 78.

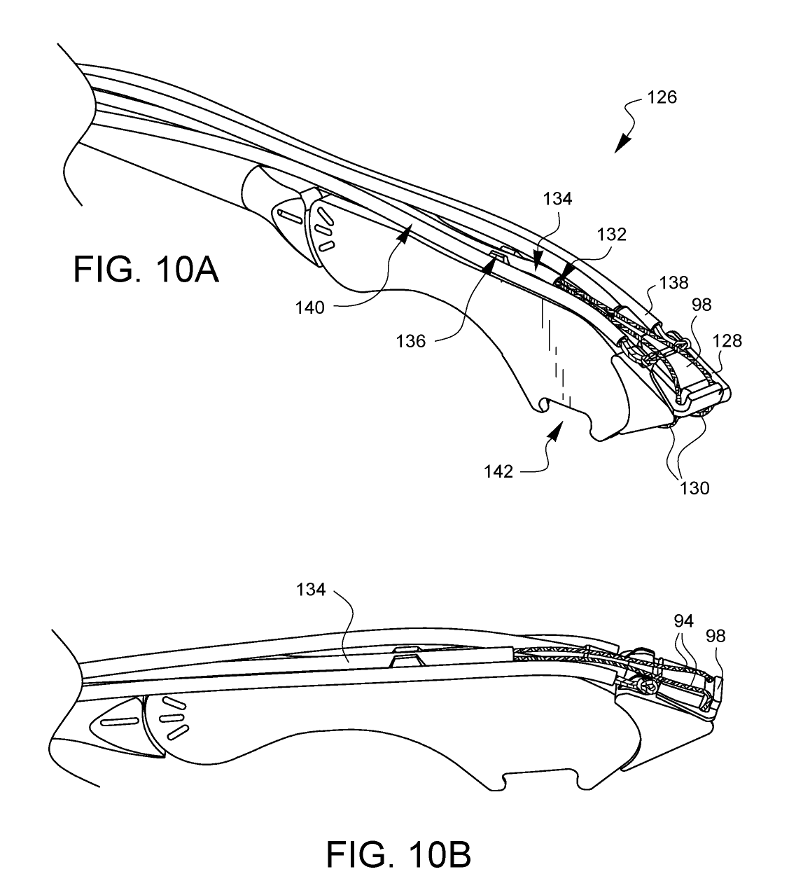

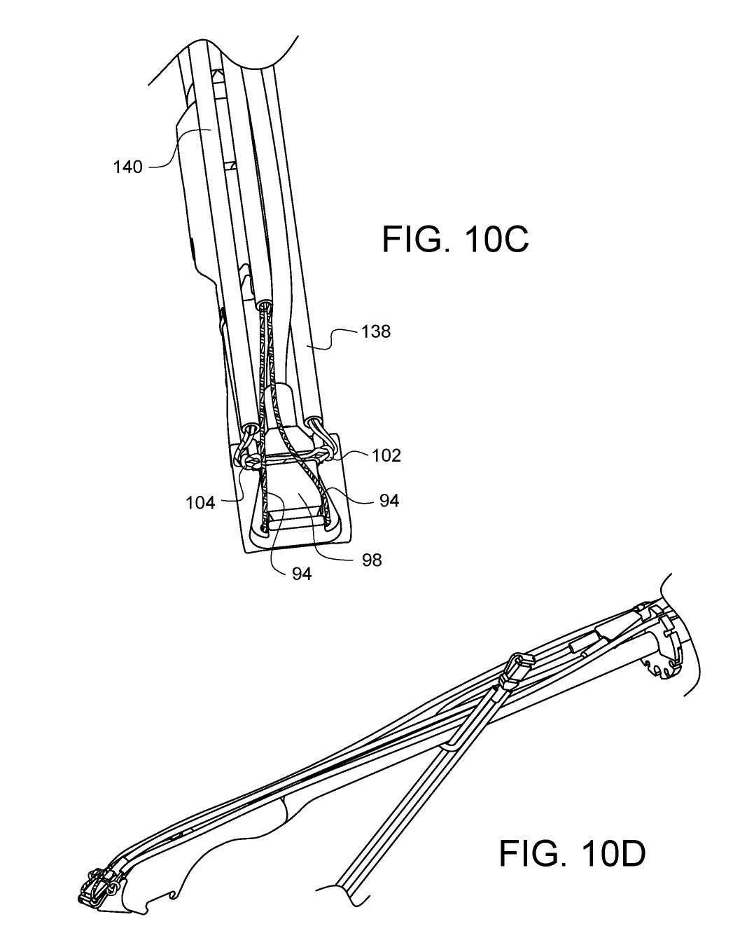

As an alternative to delivering the lead end 98 to the heart after the horizontal mattress suture stitch has been placed, a minimally invasive surgical suturing device 126 may be provided which not only places the necessary anchor stitch 90 but also simultaneously introduces the lead end to the space around the heart. The distal end of such a device 126 is shown in FIG. 10A.

A lead end rest 128 may be formed on the distal end of the suturing device 126. The ends 130 of the anchor suture 94 pass down into the suture device 126, while the middle loop 132 of the anchor suture 94 is kept organized in an anchor tube 134 that removably snaps into an anchor tube guide 136 on the back of the suturing device tip. Suture management tubes 138, 140 (which may also be referred to as coupling tubes 138, 140) are also provided for the ends of the first coupling suture 102 and the ends of the second coupling suture 104 as discussed above. The distal end of the suturing device 126 may be inserted through the lead cannula 78, and a tissue bite area 142 of the suturing device 126 may be positioned against the heart at a desired location for lead placement. The suturing operation of a suturing device to stitch a suture through the heart tissue in the tissue bite area 142 is known to those skilled in the art, while the features of the improved suturing device 126 pertaining to the lead, the configurations of anchoring suture and coupling sutures on the lead, suture management tubes, anchor tube, lead end rest, knotting to secure the pacemaker lead end, and their equivalents are not taught or suggested by the prior art. For example, see U.S. Patent Application Publication No. 2016/0345959 entitled “SUTURING DEVICE FOR MINIMALLY INVASIVE SURGERY AND NEEDLES AND METHODS THEREOF”, the specification of which is hereby incorporated by reference in its entirety. Once the anchor suture mattress stitch has been placed in the heart tissue (for example, by using a curved needle which passes through tissue in the tissue bite area 142), the anchor tube 134 can be removed from the anchor suture 134 and the suturing device 126. The tubes 138, 140 holding the coupling suture 102, 104 ends may be held in position to help hold the lead end 98 near the anchor stitch and the device tip may be withdrawn from the patient. The device tip is designed to pull the ends of the anchor sutures out with it, so the surgeon is left with two anchor suture ends (which may be removed from the suturing device) and two tubes 138, 140, each tube holding the ends of a different coupling suture 102, 104. The first coupling tube 138 may be removed and the first coupling suture 102 may be secured with one side 106 of the anchor suture 94 against the lead end 98 while it is properly oriented against the heart tissue. The second coupling tube 140 may also be removed and the second coupling suture 104 may be secured with the other side 108 of the anchor suture 94 against the lead end 98 to complete attachment of the lead end 98 to the heart. The resultant attachment will look like that of FIG. 8C.

FIGS. 10B-10D show the distal end of the minimally invasive surgical suturing device from a variety of different perspectives.

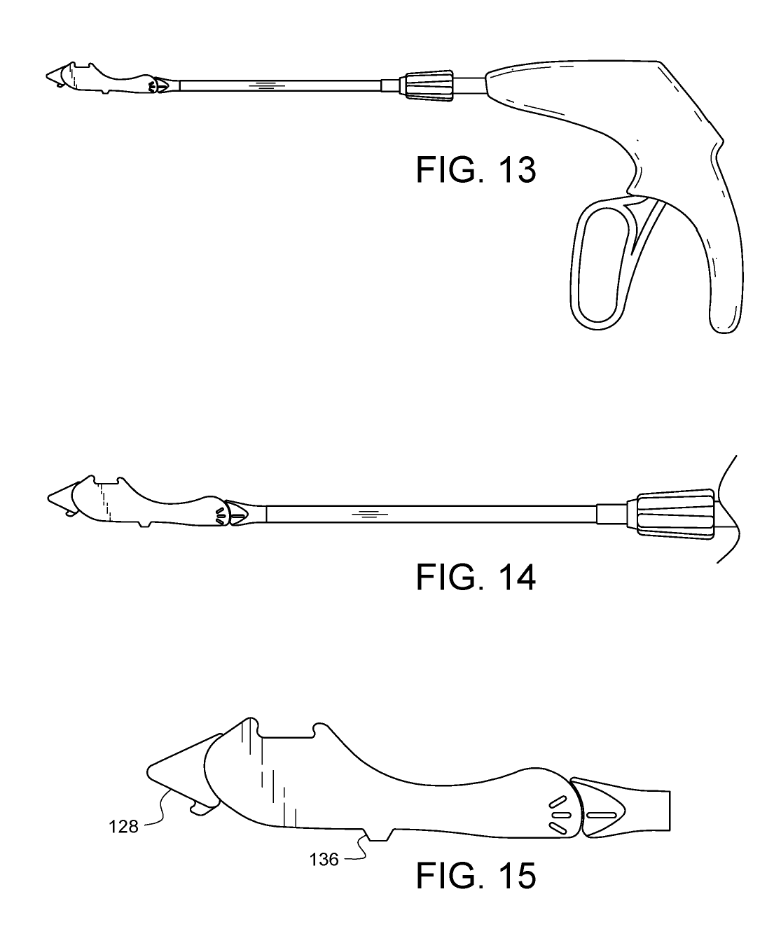

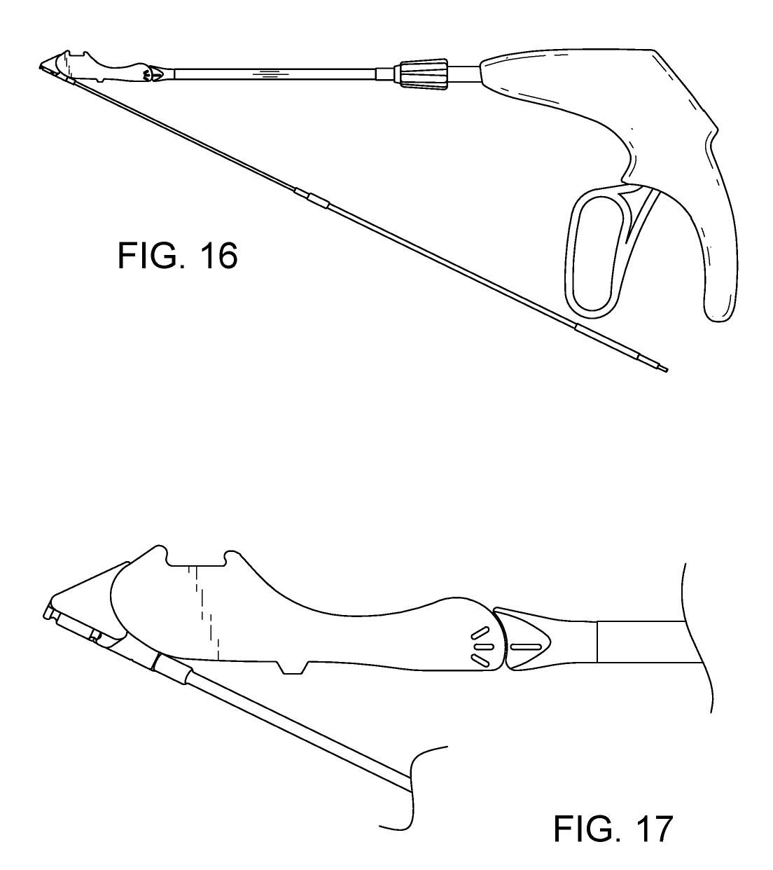

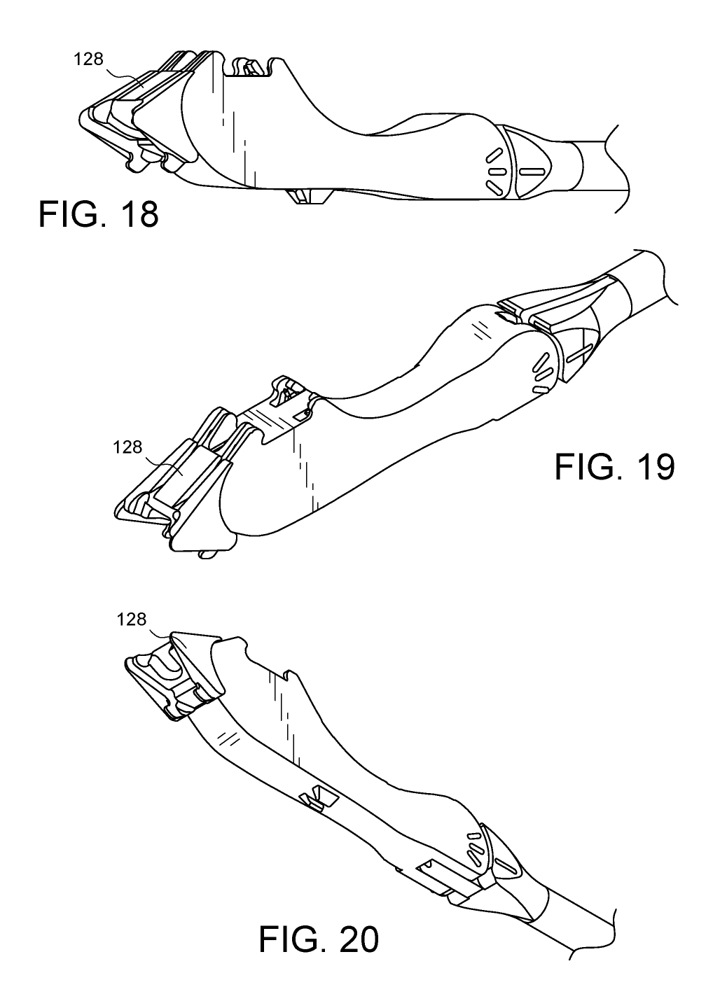

FIGS. 13-20 illustrate the surgical suturing device 126 from a variety of elevations and perspectives.

The embodiments discussed above have a wide variety of advantages over the prior art. They enable surgeons to place pacemaker leads in pediatric patients without the need for a sternotomy or thoracotomy, which will greatly reduce patient pain and recovery time. It should be understood that the concepts described herein are also applicable to non-pediatric patients. References made to pediatric leads should be interpreted as pacemaker leads which do not necessarily need to be for use in pediatric patients. The devices and methods disclosed herein, and their equivalents, make an endocardial approach unnecessary, thereby avoiding the downfalls of such procedures for lead placement. The minimally invasive tools enable surgeons to operate efficiently. Embodiments discussed herein have been described by way of example in this specification. It will be apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. The drawings included herein are not necessarily drawn to scale. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

What is claimed is:

1. A device for pacemaker lead placement, comprising:
a device tip having a tissue bite area, ferrule holders, and needle tips;
a lead end rest;
a pacemaker lead end situated on the lead end rest, the pacemaker lead end having first and second anchor suture holes;
an anchor suture with ferrules at respective ends of the anchor suture, wherein the ferrules of the anchor suture are passed through the anchor suture holes and into communication with the ferrule holders.

2. The device of claim 1, further comprising:
a first coupling suture; and
a second coupling suture.
